(19) United States
(12) Patent Application Publication
(10) Pub. No.: US 2014/0088325 A1
(43) Pub. Date: Mar. 27, 2014

(54) METHOD FOR SYNTHESIZING BETA-DICARBONYL COMPOUNDS

(71) Applicants: Stephane Honnart, Pierrefonds (FR); Philippe Galy-Jammou, Suresnes (FR)

(72) Inventors: Stephane Honnart, Pierrefonds (FR); Philippe Galy-Jammou, Suresnes (FR)

(73) Assignee: DEXERA, Paris (FR)

(21) Appl. No.: 13/824,621

(22) PCT Filed: Sep. 19, 2011
(86) PCT No.: PCT/FR2011/052143

§ 371 Date: Dec. 6, 2013

(30) Foreign Application Priority Data
Sep. 20, 2010 (FR) 1057498

(51) Int. Cl. C07C 45/61

(52) U.S. Cl. CPC C07C 45/61
USPC 568/312

(57) ABSTRACT

A method for synthesizing beta-dicarbonyl compounds, particularly beta-diketones, from at least two carbonyl compounds, such as esters and ketones, in the presence of a strong base or a mixture of strong bases by Claisen condensation with a titer of greater than 95%. The method includes providing a synthesis reactor on which a separation column, provided with a condenser and with at least one microwave generator, is mounted; feeding a first carbonyl compound and the strong base into the synthesis reactor; heating the reactor and starting up the condenser; starting up the microwave generator(s); when the mixture is brought to a boil at total flux, feeding the second carbonyl compound into the reactor; and after a waiting time, stopping the reactor and acidifying and washing the reaction mixture.)

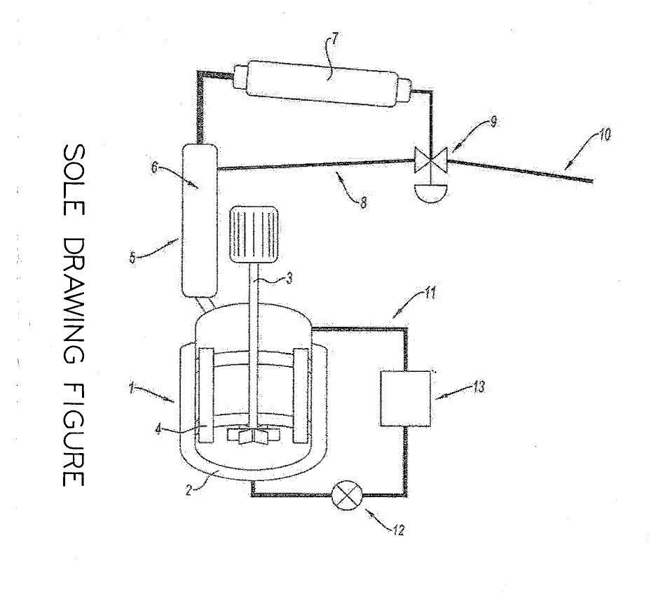

1°) The subject of this invention is a process for the industrial-scale synthesis of beta-dicarbonyl compounds from at least two carbonyl compounds such as esters or ketones, in the presence of a strong base or a mixture of strong bases, by Claisen condensation, in particular of beta-diketones from at least one ketone and at least one ester.)

1°) This process involves reacting at least two carbonyl compounds such as esters or ketones in the presence of a strong base or a mixture of strong bases, by Claisen condensation, in particular at least one ketone and at least one ester by means of the reaction:

R₁—CO—CH₂—R₂+R₃—CO—O—R₄->R₁—CO—CHR₂—CO—R₃+R₄—OH

in which
R₁, R₂ and R₃, which may be the same or different, represent a hydrogen atom, a hydrocarbon group with advantageously 1-30 carbon atoms, preferably 1-18 carbon atoms, an alkyl or alkenyl group, linear or branched with up to 24 carbon atoms, an aralkyl or cycloaraphatic group with at least 14 carbon atoms, an aralkyl group with 7-10 carbon atoms, cycloaliphatic groups that may contain double carbon-to-carbon bonds, these groups may be substituted or not, e.g. by a halogen atom or methyl or ethyl groups, or by the presence in the aliphatic chain of one or more groups with the formula: —O—, —CO—O—, —CO—, and may contain a heteroatom of oxygen or nitrogen and R₁ and R₂ may be joined in such a way that the beta-diketone forms a cycle, and in which R₄ represents an alkyl group with 1-4 carbon atoms, preferably a methyl group,

Beta-diketones are widely used additives in industrial processes as stabilising agents for plastics and cosmetic products, in particular because of their anti-UV and antioxidant properties.

For many years, compounds based on lead, cadmium and tin have been used as stabilisers in plastic materials.

However, current regulations completely ban the use of lead-based stabilisers and cadmium-based stabilising agents are currently banned in certain applications such as pipes for drinking water.

Finally, stabilising agents based on tin are going to be banned in the near future.

To replace these compounds, it has been proposed to use beta-diketones which have a number of advantages, in particular with respect to the environment.

The classic way of synthesising beta-diketones involves Claisen condensation which has been extensively reported in the literature: at least one ketone and at least one ester are reacted together in the presence of a strong base or a mixture of strong bases.

This reaction involves the formation of intermediate activated polar complexes such as enolate anions to yield beta-diketones and alcohols.

In it classically performed in a reactor containing the ester, the base (usually an alcoholate) and sometimes a solvent.

After the mixture has been heated to reflux, the ketone is added into the reactor over a matter of hours and any alcohol formed is drawn off the reaction mixture by distillation for as long as the reaction proceeds.

Extra solvent may have to be added during the reaction.

Once all the ketone has been added, time is left for completion and rest before the reaction mixture is acidified and washed, the solvent is removed and the product is purified.

One of the first descriptions of Claisen condensation is that of James M. SPRAGUE, Leland J. BECKHAM and Homer ADKINS published in December 1934 “Preparation of 1, 3 diketones by the Claisen Reaction” which describes the reaction in detail with ratios of ketone to ester ranging from 0.1 to 1.

According to this article, low molecular weight ketones (from acetone up to acetophenone) are used and the most commonly used esters are methyl acetate, methyl furoate or methyl tetrahydrofuroate. The base is elemental sodium or sodium ethanolate.

In all cases, the titre is low ranging from 15% to 70%.

The same article describes the synthesis of substituted beta-diketones from beta-diketone salts and haloalkanes.

Again, titres are relatively poor ranging from 30% to 56% and it may take up to forty hours to obtain the best titre.

Another article from March 1951 by Eugene H. MAN, Frederic W. SWAMER and Charles R. HAUSER, “The Claisen Acylation of Methyl Ketones with Branched Chain Aliphatic Esters”, proposed using a different type of base, namely sodium amide, and other ketone-ester pairs at a ketone to ester ratio of 2.

This method differs from that of SPRAGUE et al. in that the ketone is reacted with sodium amide in a solvent (ether) before the ester is added to the mixture.

However, titres are not significantly improved with reported titres of 43% to 64%.

In document U.S. Pat. No. 4,482,745 (American Cyanamid) from 1984, acetophenone and methyl benzoate are reacted together without any solvent in the presence of a divalent base, namely anhydrous lime.

Ketone, ester and lime are added to the reactor at the same time and heated to a high temperature (approaching 200° C.) with a ketone to ester ratio of between 1/1.2 and 1/10. This reaction takes 3 to 16 hours.

Adding a solvent is also suggested to promote the reaction and make it easier to process the products of the reaction.

This gives titres of between 0 and 86%. The titre was zero when the temperature was too low; when the reaction took place, titres ranged from 32% to 86%.

To get the highest titres, a great excess of ester (six times more than the ketone) and base (80% more than the ketone) were used.

An experiment using half as much base as the ketone gave a titre of 40% which supports the hypothesis that, although the valence of the base is 2, only one of these valences is used for the reaction.

Document EP0507013A1 (Witco) from 1991 proposes using a solvent and excess ester with sodium methoxide as the base.

This article focuses on the synthesis of dibenzoylmethane which is known to be a highly favourable reaction, although examples are given concerning the synthesis of related compounds; in particular, one example describes the synthesis of stearoylbenzoylmethane for which the titre was only 45%.

In all cases, given the great starting excess of ester, purification was necessary to obtain the final product.

For dibenzoylmethane, titres ranged from 84% to 95% but as soon as the compounds used were changed, the titre dropped off sharply—down to 67% for benzoyl-p-benzoylmethane and 63% for benzoyl 3,5 dimethylbenzoylmethane.

Finally, document U.S. Pat. No. 5,344,992 (Ciba) from 1994 proposes carrying the reaction out in the presence of solvents, mainly dimethyl sulfoxide (DMSO) with other co-solvents such as tetrahydrofurane or diethylene glycol dimethyl ether, and using sodium hydride as the base (or sodium methoxide in some cases).

The resultant titres were highly variable (62% to 94.5%) depending on the target compound. The highest titre was again obtained for dibenzoylmethane.

These examples show that, although it has been known for years that beta-diketones can be synthesised by Claisen condensation, the reaction is still not completely understood and titres remain highly variable.

This is largely because the reaction is a partial one resulting in an equilibrium and it proceeds in parallel to other parasite reactions. In consequence, the titre is usually relatively low and the purity of the final product obtained rarely exceeds 80%.

The best titre (95%) is obtained synthesising dibenzoylmethane but it is far lower for all the others—no more than 80%—so an extra purification step is required.

However, this supplementary step has major negative environmental impact because it requires large volumes of solvent and a great deal of energy as well as generating residues of impurities which have to be disposed of.

By way of example, separating the impurities out of an 80% pure product means a loss of 20% of the product itself to obtain a final titre of 95%.

Separating the impurities out of an 60% pure product means a loss of nearly 47% of the product itself to obtain a final titre of 95%.

Taking all this together, the classic synthetic method for beta-diketones based on Claisen condensation is subject to major loss of the product, great expense and substantial adverse environmental impact.

Specifically, the disadvantage results from the fact that, to inhibit parasite reactions (mainly ester and ketone self-condensation reactions), the reaction mixture has to be as uniform as possible in temperature and concentration and the alcohol has to be evaporated off as quickly as possible as it is formed.

It has been shown that if there is no alcohol present in the reaction mixture, the equilibrium of the Claisen reaction is shifted away from parasite reactions to favour the synthesis of beta-dicarbonyl compounds.

However, to ensure fast evaporation of the alcohol, enough energy per unit volume has to be delivered into the reaction mixture. While this is not a problem in the laboratory, it complicates industrial-scale production for which the reactors are large and usually fitted with a double-jacket containing a heat-conducting fluid as well as a mixing system. The volume of these reactors increases as the cube of the diameter while the heating surface only increases as the square. In consequence, the surface-to-volume ratio—which conditions the amount of energy delivered per unit volume of the reactor—decreases in direct proportion to the diameter of the reactor.

Thus, scaling up by a factor of 1,000 (a classic scaling factor between laboratory and small industrial-scale production) results in a ten-fold decrease in the energy delivered per unit volume.

To solve this problem and increase the amount of energy delivered into the industrial reaction mixture to bring it to the level achieved in the laboratory, engineers specialising in the design of equipment for industrial chemical synthesis have already tested three possible solutions to the problem, i.e. how to increase the temperature difference between the heat-conducting fluid and the reaction mixture. These are adding a heating coil inside the reactor and adding a recirculation loop inside the reactor driven by a pump and fitted with a heat-exchanger. However, none of these gave the desired result.

This is due to the fact that the reaction mixture is heated by thermal conduction across the reactor wall or the heat exchanger, and then by forced convection which occurs because there is a steep temperature gradient between the fluid in the middle of the reactor and that near the walls.

However, this temperature gradient entails local parasite reactions to such an extent that the overall titre of the reaction is substantially affected.

In particular, an increase in the temperature difference between heat-conducting fluid and reaction mixture leads to local heating at the reactor walls and causes breakdown of the reagents and induces parasite reactions.

Similarly, the presence of a heating coil inside the reactor alters flow inside the reaction mixture in such a way as to compromise its turnover at the reactor surface and therefore inhibit alcohol evaporation—which itself encourages parasite reactions.

Substituting the heating coil with a heat-exchanger mounted on an external recirculation loop did not work any better since flow through the pipe can never come close to matching that generated by the mixing system.

For a standard 10 m³ reactor, it is difficult to imagine exceeding a recirculation rate of over 50-100 m³/hour while a regular mixing system will generate flow rates of up to 1,000 m³/hour, for two reasons.

The first is due to safety concerns: if the reaction mixture is flowing too fast through the pipes of the external recirculation circuit, there is a risk of explosion due to build-up of electrical charge.

The second reason is related to hydrodynamic conditions inside the reactor: a recirculation rate of anything over about ten volumes per hour will compromise flow induced by the mixing system.

As a result, there is not currently any process for synthesising beta-dicarbonyl compounds at an industrial scale that ensures the delivery of enough energy per unit volume of reaction mixture to drive sufficiently fast evaporation of the alcohol formed in the reaction.

The subject of this invention is to overcome this problem by proposing a process for the synthesis of beta-dicarbonyl compounds—in particular beta-diketones—by Claisen condensation that guarantees a reaction mixture uniform in terms of both temperature and concentration at the same time as very rapid evaporation of the alcohol as it is formed in the reaction.

According to the invention, this process enhances the titre of the reaction and the purity of the product obtained, in particular a titre of over 95% and notably one of over 98%, i.e. a titre never hitherto achieved for this type of reaction, so there is no need to purify the final product.

The process according to the invention is therefore particularly advantageous from both the economic and the environmental points of view.

According to the invention, this process is characterised by the following steps:
a synthesis reactor is assembled, preferably with a double jacket, topped with a separating column fitted with a condenser with variable reflux controlled by the column temperature, and fitted with at least one source of microwaves and a mixing system,
a first carbonyl compound is introduced with the strong base into the reactor, with mixing,
the reactor is heated and the condenser is turned on,
the microwave source or sources are turned on,
once the mixture is boiling with total reflux in the head of the separating column, the second carbonyl compound is added to the reactor and
after an interval, the reactor is turned off and the reaction mixture is acidified and washed.

It should be noted that, according to the invention, the reactor can be fitted with at least one microwave generator directly mounted for example on flanges inside, in particular at its sky level, and/or notably if there is insufficient space here, at least one external microwave generator connected via a wave guide to direct the microwaves into the reaction mixture, and/or also fitted with an external recirculation loop fitted with a recirculating pump and a microwave generator.

It should be noted that choice of the number and nature of microwave generators associated with the reactor means that the energy per unit volume delivered into the reaction mixture can be perfectly controlled.

The essential characteristic of the process according to the invention is thus the use of microwave energy to heat the reaction mixture.

To a great extent, this eliminates parasite reactions, in particular self-condensation reactions between the reagents, by increasing the energy density in the reaction mixture and enhancing the uniformity of the mixture in terms of temperature and concentration, thereby considerably raising the titre of the resultant product.

As a corollary, using microwaves cuts down reaction times, notably by a factor of at least two compared with the classic process, and in parallel massively enhances productivity, easily by a factor of up to five.

The process according to the invention is therefore particularly advantageous from the economic and environmental points of view by virtue of the reduction in raw materials consumption; it is also advantageous in terms of safety and investment costs because of the reductions in equipment size and reaction time.

These advantages follow on from the fact that, in the framework of the process according to the invention, the microwaves mainly act in two ways: the first is related to how energy is delivered into the reaction mixture while the second is related to vibrational effects.

In practice, the way microwaves heat the reaction mixture is completely different from the classic method in that the energy is delivered into the heart of the medium and the temperature at the hot point is only slightly higher than the mean temperature throughout the reactor.

In consequence, the reactions that occur throughout the reactor are uniform and can be optimised to ensure a higher titre.

The second mechanism of action of the microwaves is associated with their vibrational effects: the activated, polar intermediate complexes that form in the course of Claisen condensation create a significant energetic barrier that has to be overcome if the reaction is to proceed.

However, it has been shown that the vibrations induced by the microwaves stabilise these energetic complexes, depleting their energy and thereby improving the kinetics of the reaction with less effect on the parasite reactions.

This too then is a positive effect which further cuts down reaction time.

It should be noted that using microwaves has already been proposed to speed up chemical reactions, the rate of which often depends on the temperature of the reaction mixture.

However, a substantial temperature rise entails a sharp rise in pressure—possibly up to 20 bar—which is possible with laboratory equipment but is difficult to scale up for industrial-scale production.

At equivalent pressure, the only gain observed to date is the possibility of heating the reaction mixture up faster.

For this reason, using microwaves is quite common in laboratories where whole series of experiments with short turnover times have to be conducted, in particular to validate reagents.

For this, products to be tested are classically added to test tubes that can be pressurised and they are then heated in a microwave oven in order to speed the reaction up.

However, speeding reactions up with microwaves in this way is of limited interest at the industrial scale because the time factor is less key and it would be expensive.

In practice, the loss of time associated with using classic heating systems is more than compensated by the savings on microwave apparatus and on energy costs because generating microwaves consumes electricity which is far more expensive than for example classic steam-heating with a combustion boiler.

On the other hand, microwaves are ideal in the framework of this invention in which the excess energy and investment costs will be largely compensated for by the possibility of obtaining a very high titre and thereby avoiding the need for subsequent purification steps.

The first step in the process according to the invention therefore consists of assembling the reactor in which the Claisen condensation reaction is to be carried out.

An example of such a synthesis reactor is illustrated in the non-limiting drawing appended herewith.

According to this drawing, the synthesis reactor 1 consists of a double jacketed chamber 2 fitted with a mixing system 3 and counter-blades.

At the top of this reactor 1, there is a separating column 5 connected to a condenser 7 and a backflow pipe 8.

The separating column 5 is fitted with a temperature sensor 6 which controls a regulatory valve/stopcock 9 to control what fraction of the condensed liquid returned to the column 5 via the backflow pipe 8 or is drawn off through a drain pipe 10, depending on the temperature.

The reactor synthesis 1 is also fitted with an external recirculating loop 11 fitted with a pump 12 and a microwave source 13.

According to a particularly advantageous characteristic of the invention, the carbonyl compounds consist of at least one ketone and at least one ester.

It should be noted that, according to the invention, the reaction can be selectively run with stoichiometric proportions of these two reagents or with either the ester or ketone in molar excess, obtaining a beta-diketone titre of over 95% in all cases.

The possibility of using excess ketone corresponds to a special advantage of the process according to the invention over classic processes for the industrial-scale synthesis of beta-diketones by Claisen condensation in which the ester always has to be in molar excess over the ketone.

In practice, when the reaction mixture is not uniform in composition and/or temperature and if the alcohol is not effectively removed from the medium as it is formed, spots form where the local ketone concentration is high and this leads to extensive self-condensation of this raw material, drastically reducing both the yield of the reaction and the purity of the final product as well as necessitating an extra purification step which further reduces the amount of useful product.

In contrast, the process according to the invention allows reaction conditions in which the ketone is in molar excess over the ester so the latter is almost all converted with only very minor contamination of the final product.

Moreover, if the ester is expensive and the ketone cheap, the process according to the invention affords savings by virtue of being able to use more ketone than ester, above and beyond the savings resulting from the increased purity of the final product.

In consequence, compared with classic industrial synthesis processes, it is when the ketone is in molar excess that using microwaves according to the invention affords the greatest improvement in yield.

According to another characteristic of the invention, the conjugate acid of the strong base used is volatile in the conditions of the reaction, e.g. an alcoholate, notably an alcoholate of sodium and in particular sodium methoxide.

According to the invention, the reaction conditions can be substantially manipulated depending on the starting products and the type of beta-dicarbonyl compound to be synthesised, in particular the type of beta-diketone.

The process according to the invention can in particular be run without any solvent or with a pure or mixed solvent, notably a solvent with an aromatic core.

The reaction can be run in a vacuum or at any pressure, notably atmospheric pressure or a lower pressure of 0-1 atmosphere absolute, preferably 0.1-0.5 atmosphere absolute, or alternatively at a higher pressure from 0-5 relative atmospheres, preferably 0-2 relative atmospheres.

Moreover and again depending on the starting products and the type of beta-dicarbonyl compound to be synthesised, in particular the type of beta-diketone, the temperature of the reaction can be located within a range of 60-180° C., preferably between 90° C. and 140° C.

It is also advantageous according to the invention to render the reactor inert with nitrogen gas at the beginning of synthesis and maintain a gentle flow of the gas through the reactor throughout the reaction.

The characteristics and advantages of the process according to the invention—in particular those related to the use of microwaves—will be easier to understand in the light of the following examples:

### EXAMPLE 1

### Synthesis of StearoylBenzoylMethane (SBM) using the “Classic” Process

In a classic glass, double-jacketed chemical engineering reactor with a volume of 1 litre with an effective mixing system, add 450 mL xylenes, 178.79 g fused methyl stearate and 34.05 g powdered sodium methoxide. Once the reagents have been added, render the reactor inert with a continuous flow of nitrogen gas. Then bring the mixture to boiling point and complete reflux at the head of the separating column. Add 68.42 g acetophenone over 5 hours.

Throughout the five-hour reaction, draw the methanol off at the head of the separating column. Once all the acetophenone has been added, allow the reaction to run for about one more hour. After this extra hour, acidify the mixture and then wash it. Analyse the resultant organic solution by gas phase chromatography. Almost all the acetophenone is converted and the SBM titre is 82.5%.

SBM productivity during the reaction phase is 30.3 kg/h/m3.

### EXAMPLE 2

### Synthesis of StearoylBenzoylMethane (SBM) using the Process According to the Invention

The experimental apparatus consists of a classic glass, double jacketed chemical engineering reactor with a volume of 1 litre with an effective mixing system. This is topped with a separating column fitted with a variable-reflux condenser. It also has a recirculating loop fitted with a gear-type pump and a 600 W microwave generator.

Add 450 mL xylenes, 178.82 g fused methyl stearate and 34.02 g powdered sodium methoxide. Once the reagents have been added, render the reactor inert with a continuous flow of nitrogen gas. Recirculate the mixture through the external circuit at a rate of 15 kg/h. Bring to boiling and total reflux, and then switch the microwave source on.

Add 68.39 g acetophenone over one hour. Once it has all been added, let the reaction continue for another 15 minutes. Throughout all this time, draw the methanol off the reaction mixture. After the 15 minutes of finishing time, switch off the microwave source and the heater. Acidify the mixture and then wash it.

Analysis by gas phase chromatography shows that almost all the acetophenone is consumed and that the SBM titre is 98.1%.

The titre of SBM obtained by the process according to the invention is more than 15 percentage points better than with the classic process. SBM productivity during the reaction phase is 172.6 kg/h/m3, i.e. 5.7 times that obtained in the classic process.

### EXAMPLE 3

### Synthesis of StearoylBenzoylMethane (SBM) using the Process According to the Invention Without a Microwave Source

The experimental apparatus consists of a classic glass, double jacketed chemical engineering reactor with a volume of 1 litre with an effective mixing system. This is topped with a separating column fitted with a variable-reflux condenser. It is also fitted with a double jacketed recirculation loop with a gear-type pump. The jacket temperature is kept very high to try to transfer as much heat as in Example 2.

Add 450 mL xylenes, 178.77 g fused methyl stearate and 34.00 g powdered sodium methoxide. Once the reagents have been added, render the reactor inert with a continuous flow of nitrogen gas. Recirculate the mixture through the external circuit at a rate of 15 kg/h. Bring to boiling and complete reflux.

Add 68.41 g acetophenone over one hour. Once it has all been added, let the reaction continue for another 15 minutes. Throughout all this time, draw the methanol off the reaction mixture. After the 15 minutes of finishing time, switch off the heater. Acidify the mixture and then wash it.

The organic phase in intensely coloured. Analysis by gas phase chromatography shows that almost all the acetophenone is consumed and that the SBM titre is 71.8%. The chromatogram shows a whole series of peaks corresponding to various parasite reactions.

### EXAMPLE 4

### Synthesis of DiBenzoylMethane (DBM) using the Process According to the Invention

The experimental apparatus consists of a classic glass, double jacketed chemical engineering reactor with a volume of 1 litre with an effective mixing system. This is topped with a separating column fitted with a variable-reflux condenser. It also has a recirculating loop fitted with a gear-type pump and a 600 W microwave generator.

Add 560 mL xylenes, 81.59 g fused methyl benzoate and 34.03 g powdered sodium methoxide. Once the reagents have been added, render the reactor inert with a continuous flow of nitrogen gas. Recirculate the mixture through the external circuit at a rate of 15 kg/h. Bring to boiling and total reflux, and then switch the microwave source on.

Add 68.42 g acetophenone over one hour. Once it has all been added, let the reaction continue for another 15 minutes. Throughout all this time, draw the methanol off the reaction mixture. After the 15 minutes of finishing time, switch off the microwave source and the heater. Acidify the mixture and then wash it.

Analysis of the organic phase by gas phase chromatography shows that almost all the acetophenone is consumed and that the DBM titre is 99.2%. DBM productivity during the reaction phase is 101.4 kg/h/m3.

### EXAMPLE 5

### Synthesis of DiBenzoylMethane (DBM) using the Process According to the Invention with No Solvent

The experimental apparatus consists of a classic glass, double jacketed chemical engineering reactor with a volume of 1 litre with an effective mixing system. This is topped with a separating column fitted with a variable-reflux condenser. It also has a recirculating loop fitted with a gear-type pump, a 600 W microwave generator and a vacuum pump capable of reducing the pressure of the system to about 100 mbar.

Add 683.52 g fused methyl benzoate and 34.00 g powdered sodium methoxide. Once the reagents have been added, render the reactor inert with a slow continuous flow of nitrogen gas maintaining a partial vacuum at about 300 mbar. Recirculate the mixture through the external circuit at a rate of 15 kg/h. Bring to boiling and total reflux, and then switch the microwave source on.

Add 68.40 g acetophenone over one hour. Once it has all been added, let the reaction continue for another 15 minutes. Throughout all this time, draw the methanol off the reaction mixture. After the 15 minutes of finishing time, switch off the microwave source and the heater. Acidify the mixture and then wash it.

Analysis of the organic phase by gas phase chromatography shows that almost all the acetophenone is consumed and that the DBM titre is 99.7%. DBM productivity during the reaction phase is 101.8 kg/h/m3.

### EXAMPLE 6

### Synthesis of StearoylBenzoylMethane (SBM) using the Process According to the Invention

The experimental apparatus consists of a classic glass, double jacketed chemical engineering reactor with a volume of 1 litre with an effective mixing system. This is topped with a separating column fitted with a variable-reflux condenser. It also has a recirculating loop fitted with a gear-type pump and a 600 W microwave generator.

Add 440 mL xylenes, 178.76 g fused methyl stearate and 42.87 g powdered sodium methoxide. Once the reagents have been added, render the reactor inert with a continuous flow of nitrogen gas. Recirculate the mixture through the external circuit at a rate of 15 kg/h. Bring to boiling and total reflux, and then switch the microwave source on.

Add 68.45 g acetophenone over one hour. Once it has all been added, let the reaction continue for another 15 minutes. Throughout this time, draw any methanol and ethanol generated off the reaction mixture. After the 15 minutes of finishing time, switch off the microwave source and the heater. Acidify the mixture and then wash it.

Analysis by gas phase chromatography shows that almost all the aceto-phenone is consumed and that the SBM titre is 98.3%.

SBM productivity during the reaction phase is 173.2 kg/h/m3.

### EXAMPLE 7

### Synthesis of OctanoylBenzoylMethane (OBM) using the Process According to the Invention

The experimental apparatus consists of a classic glass, double jacketed chemical engineering reactor with a volume of 1 litre with an effective mixing system. This is topped with a separating column fitted with a variable-reflux condenser. It also has a recirculating loop fitted with a gear-type pump and a 600 W microwave generator.

Add 550 mL xylenes, 94.78 g fused methyl octanoate and 34.05 g powdered sodium methoxide. Once the reagents have been added, render the reactor inert with a continuous flow of nitrogen gas. Recirculate the mixture through the external circuit at a rate of 15 kg/h. Bring to boiling and total reflux, and then switch the microwave source on.

Add 68.41 g acetophenone over one hour. Once it has all been added, let the reaction continue for another 15 minutes. Throughout all this time, draw the methanol off the reaction mixture. After the 15 minutes of finishing time, switch off the microwave source and the heater. Acidify the mixture and then wash it.

Analysis of the organic phase by gas phase chromatography shows that almost all the acetophenone is consumed and that the OBM titre is 98.3%. OBM productivity during the reaction phase is 110.3 kg/h/m3.

### EXAMPLE 8

### Synthesis of StearoylBenzoylMethane (SBM) using the Process According to the Invention with Excess Ketone

The experimental apparatus consists of a classic glass, double jacketed chemical engineering reactor with a volume of 1 litre with an effective mixing system. This is topped with a separating column fitted with a variable-relux condenser. It also has a recirculating loop fitted with a gear-type pump and a 600 W microwave generator.

Add 450 mL xylenes, 178.81 g fused methyl stearate and 34.02 g powdered sodium methoxide. Once the reagents have been added, render the reactor inert with a continuous flow of nitrogen gas. Recirculate the mixture through the external circuit at a rate of 15 kg/h. Bring to boiling and total reflux, and then switch the microwave source on.

Add 73.19 g acetophenone over one hour. Once it has all been added, let the reaction continue for another 15 minutes. Throughout all this time, draw the methanol off the reaction mixture. After the 15 minutes of finishing time, switch off the microwave source and the heater. Acidify the mixture and then wash it.

Analysis by gas phase chromatography shows that almost all the aceto-phenone is consumed and that the SBM titre is 97.5% compared with the ester.

SBM productivity during the reaction phase is 180.6 kg/h/m3.

### EXAMPLE 9

### Synthesis of PalmitoylBenzoylMethane (PBM) using the Process According to the Invention

The experimental apparatus consists of a classic glass, double jacketed chemical engineering reactor with a volume of 1 litre with an effective mixing system. This is topped with a separating column fitted with a variable-reflux condenser. It also has a recirculating loop fitted with a gear-type pump and a 600 W microwave generator.

Add 470 mL xylenes, 159.99 g fused methyl palmitate and 34.03 g powdered sodium methoxide. Once the reagents have been added, render the reactor inert with a continuous flow of nitrogen gas. Recirculate the mixture through the external circuit at a rate of 15 kg/h. Bring to boiling and total reflux, and then switch the microwave source on.

Add 68.40 g acetophenone over one hour. Once it has all been added, let the reaction continue for another 15 minutes. Throughout all this time, draw the methanol off the reaction mixture. After the 15 minutes of finishing time, switch off the microwave source and the heater. Acidify the mixture and then wash it.

Analysis of the organic phase by gas phase chromatography shows that almost all the acetophenone is consumed and that the PBM titre is 98.0%.

PBM productivity during the reaction phase is 168.4 kg/h/m3.

### EXAMPLE 10

### Synthesis of MyristoylBenzoylMethane (MBM) using the Process According to the Invention

The experimental apparatus consists of a classic glass, double jacketed chemical engineering reactor with a volume of 1 litre with an effective mixing system. This is topped with a separating column fitted with a variable-reflux condenser. It also has a recirculating loop fitted with a gear-type pump and a 600 W microwave generator.

Add 490 mL xylenes, 145.22 g fused methyl myristate and 33.98 g powdered sodium methoxide. Once the reagents have been added, render the reactor inert with a continuous flow of nitrogen gas. Recirculate the mixture through the external circuit at a rate of 15 kg/h. Bring to boiling and total reflux, and then switch the microwave source on.

Add 68.36 g acetophenone over one hour. Once it has all been added, let the reaction continue for another 15 minutes. Throughout all this time, draw the methanol off the reaction mixture. After the 15 minutes of finishing time, switch off the microwave source and the heater. Acidify the mixture and then wash it.

Analysis of the organic phase by gas phase chromatography shows that almost all the acetophenone is consumed and that the MBM titre is 98.1%.

MBM productivity during the reaction phase is 155.4 kg/h/m3.

### EXAMPLE 11

### Synthesis of LauroylBenzoylMethane (LBM) using the Process According to the Invention

The experimental apparatus consists of a classic glass, double jacketed chemical engineering reactor with a volume of 1 litre with an effective mixing system. This is topped with a separating column fitted with a variable-reflux condenser. It also has a recirculating loop fitted with a gear-type pump and a 600 W microwave generator.

Add 510 mL xylenes, 128.42 g fused methyl laurate and 34.01 g powdered sodium methoxide. Once the reagents have been added, render the reactor inert with a continuous flow of nitrogen gas. Recirculate the mixture through the external circuit at a rate of 15 kg/h. Bring to boiling and total reflux, and then switch the microwave source on.

Add 68.41 g acetophenone over one hour. Once it has all been added, let the reaction continue for another 15 minutes. Throughout all this time, draw the methanol off the reaction mixture. After the 15 minutes of finishing time, switch off the microwave source and the heater. Acidify the mixture and then wash it.

Analysis of the organic phase by gas phase chromatography shows that almost all the acetophenone is consumed and that the LBM titre is 98.3%.

LBM productivity during the reaction phase is 142.5 kg/h/m3.

### EXAMPLE 12

### Synthesis of DecanoylBenzoylMethane (DeBM) using the Process According to the Invention

The experimental apparatus consists of a classic glass, double jacketed chemical engineering reactor with a volume of 1 litre with an effective mixing system. This is topped with a separating column fitted with a variable-reflux condenser. It also has a recirculating loop fitted with a gear-type pump and a 600 W microwave generator.

Add 530 mL xylenes, 111.58 g fused methyl decanoate and 34.00 g powdered sodium methoxide. Once the reagents have been added, render the reactor inert with a continuous flow of nitrogen gas. Recirculate the mixture through the external circuit at a rate of 15 kg/h. Bring to boiling and total reflux, and then switch the microwave source on.

Add 68.45 g acetophenone over one hour. Once it has all been added, let the reaction continue for another 15 minutes. Throughout all this time, draw the methanol off the reaction mixture. After the 15 minutes of finishing time, switch off the microwave source and the heater. Acidify the mixture and then wash it.

Analysis of the organic phase by gas phase chromatography shows that almost all the acetophenone is consumed and that the DeBM titre is 98.3%.

DeBM productivity during the reaction phase is 129.3 kg/h/m3.

### EXAMPLE 13

### Synthesis of Benzoyl p-MethylBenzoylMethane (BpMBM) Using the Process According to the Invention

The experimental apparatus consists of a classic glass, double jacketed chemical engineering reactor with a volume of 1 litre with an effective mixing system. This is topped with a separating column fitted with a variable-reflux condenser. It also has a recirculating loop fitted with a gear-type pump and a 600 W microwave generator.

Add 550 mL xylenes, 90.02 g fused methyl benzoate and 34.02 g powdered sodium methoxide. Once the reagents have been added, render the reactor inert with a continuous flow of nitrogen gas. Recirculate the mixture through the external circuit at a rate of 15 kg/h. Bring to boiling and total reflux, and then switch the microwave source on.

Add 68.42 g acetophenone over one hour. Once it has all been added, let the reaction continue for another 15 minutes. Throughout all this time, draw the methanol off the reaction mixture. After the 15 minutes of finishing time, switch off the microwave source and the heater. Acidify the mixture and then wash it.

Analysis of the organic phase by gas phase chromatography shows that almost all the acetophenone is consumed and that the BpMBM titre is 98.8%.

BpMBM productivity during the reaction phase is 112.9 kg/h/m3.

### EXAMPLE 14

### Synthesis of Benzoyl 3,5-DiMethylBenzoylMethane (BDMBM) Using the Process According to the Invention

The experimental apparatus consists of a classic glass, double jacketed chemical engineering reactor with a volume of 1 litre with an effective mixing system. This is topped with a separating column fitted with a variable-reflux condenser. It also has a recirculating loop fitted with a gear-type pump and a 600 W microwave generator.

Add 560 mL xylenes, 81.59 g fused methyl benzoate and 34.01 g powdered sodium methoxide. Once the reagents have been added, render the reactor inert with a continuous flow of nitrogen gas. Recirculate the mixture through the external circuit at a rate of 15 kg/h. Bring to boiling and total reflux, and then switch the microwave source on.

Add 84.35 g 3,5-dimethylacetophenone over one hour. Once it has all been added, let the reaction continue for another 15 minutes. Throughout all this time, draw the methanol off the reaction mixture. After the 15 minutes of finishing time, switch off the microwave source and the heater.

Acidify the mixture and then wash it.

Analysis of the organic phase by gas phase chromatography shows that almost all the 3,5-dimethylacetophenone is consumed and that the BDMBM titre is 98.6%.

BDMBM productivity during the reaction phase is 119.2 kg/h/m3.

### EXAMPLE 15

### Industrial-scale synthesis of StearoylBenzoylMethane (SBM) Using the Process According to the Invention (1 m³).

The industrial set-up consists of a classic stainless steel, double-jacketed chemical engineering reactor with a volume of 1,000 litres with an effective mixing system. This is topped with a separating column fitted with a variable-reflux condenser. It is also fitted with enough microwave sources to ensure a global power output of 30 kW.

Add 450 litres of xylenes, 178.9 kg fused methyl stearate and 33.95 kg powdered sodium methoxide. Once the reagents have been added, bring the mixture to boiling and complete reflux, and turn the microwave generators on.

Add 68.5 kg acetophenone over two hours. Once it has all been added, let the reaction continue for another 30 minutes. Throughout all this time, draw the methanol off the reaction mixture. After the 30 minutes of finishing time, switch off the microwave source and the heater. Acidify the mixture and then wash it.

Analysis by gas phase chromatography shows that almost all the aceto-phenone is consumed and that the SBM titre is 97.6%.

### EXAMPLE 16

### Industrial-scale synthesis of StearoylBenzoylMethane (SBM) Using the Process According to the Invention (10 m³).

The industrial set-up consists of a classic stainless steel, double-jacketed chemical engineering reactor with a volume of 10,000 litres with an effective mixing system. This is topped with a separating column fitted with a variable-reflux condenser. It is also fitted with enough microwave sources to ensure a global power output of 120 kW.

Add 4,500 litres of xylenes, 1,788.6 kg fused methyl stearate and 340 kg powdered sodium methoxide. Once the reagents have been added, bring the mixture to boiling and complete reflux, and turn the microwave generators on.

Add 684 kg acetophenone over four hours. Once it has all been added, let the reaction continue for another 30 minutes. Throughout all this time, draw the methanol off the reaction mixture. After the 30 minutes of finishing time, switch off the microwave source and the heater. Acidify the mixture and then wash it.

Analysis by gas phase chromatography shows that almost all the aceto-phenone is consumed and that the SBM titre is 97.2%.

1-11. (canceled)

12. A process for the industrial-scale synthesis of beta-dicarbonyl compounds from at least two carbonyl compounds such as esters or ketones, in the presence of a strong base or a mixture of strong bases, by Claisen condensation with a titre of over 95%, in particular of beta-diketones from at least one ketone and at least one ester by means of the reaction:
R₁—CO—CH₂—R₂+R₃—CO—O—R₄->R₁—CO—CHR₂—CO—R₃+R₄—OH
in which R₁, R₂ and R₃, which may be the same or different, represent a hydrogen atom, a hydrocarbon group with advantageously 1-30 carbon atoms, preferably 1-18 carbon atoms, an alkyl or alkenyl group, linear or branched with up to 24 carbon atoms, an aralkyl or cycloaraphatic group with at least 14 carbon atoms, an aralkyl group with 7-10 carbon atoms, cycloaliphatic groups that may contain double carbon-to-carbon bonds, these groups may be substituted or not, e.g. by a halogen atom or methyl or ethyl groups, or by the presence in the aliphatic chain of one or more groups with the formula: —O—, —CO—O—, —CO—, and may contain a heteroatom of oxygen or nitrogen and R₁ and R₂ may be joined in such a way that the beta-diketone forms a cycle, and in which R₄ represents an alkyl group with 1-4 carbon atoms, preferably a methyl group, characterised by the following steps:
assembling a synthesis reactor, including a double jacket topped with a separating column having a condenser with variable reflux controlled by the column temperature, and further including at least one source of microwaves and a mixing system;
introducing a first carbonyl compound with the strong base into the reactor, with mixing;
heating the reactor and turning on the condenser;
turning on the microwave source or sources;
adding the second carbonyl compound to the reactor once the mixture is boiling with total reflux in the separating column; and
turning off the reactor and acidifying and washing the reaction mixture, after an interval.

13. The process of claim 12, wherein the reactor includes at least one microwave source directly mounted inside the reactor and an external microwave source connected to the reactor via a wave guide to conduct the microwaves into the reaction mixture.

14. The process of claim 12, wherein the reactor includes an external recirculating loop having a pump and a microwave source.

15. The process of claim 12, wherein the carbonyl compounds comprises at least one ketone and at least one ester.

16. The process of claim 15, wherein the ketone is in molar excess compared with the ester.

17. The process of claim 12, wherein the conjugate acid of the strong base is volatile in the conditions of the reaction.

18. The process of claim 17, wherein the strong base is an alcoholate.

19. The process of claim 12, wherein the reaction temperature is between 60-180° C.

20. The process of claim 12, wherein the reaction is conducted in the absence of any solvent.

21. The process of claim 12, wherein the reaction is conducted in the presence of one of a pure solvent and a mixed solvent.

22. The process of claim 12, wherein a gentle flow of nitrogen gas is maintained in the reactor throughout the reaction.

23. The process of claim 13, wherein the reactor includes an external recirculating loop having a pump and a microwave source.

24. The process of claim 14, wherein the carbonyl compounds comprise at least one ketone and at least one ester.

25. The process of claim 13, wherein the conjugate acid of the strong base is volatile in the conditions of the reaction.

26. The process of claim 14, wherein the conjugate acid of the strong base is volatile in the conditions of the reaction.

27. The process of claim 15, wherein the conjugate acid of the strong base is volatile in the conditions of the reaction.

28. The process of claim 13, wherein the reaction temperature is between 60-180° C.

29. The process of claim 14, wherein the reaction temperature is between 60-180° C.

30. The process of claim 18, wherein the strong base is sodium methylate.

31. The process of claim 19, wherein the reaction temperature is between 90° C. and 140° C.
